(19) United States
(12) Patent Application Publication
(10) Pub. No.: US 2024/0285572 A1
(43) Pub. Date: Aug. 29, 2024

(54) COMPOSITIONS COMPRISING NITRIC OXIDE-RELEASING COMPOUNDS FOR TREATING CHOROIDAL VASCULAR DISEASES AND METHODS OF THEIR USE

(71) Applicant: OPTICGON LLC, Flushing, NY (US)

(72) Inventor: Ian H. CHAN, Flushing, NY (US)

(21) Appl. No.: 18/587,768

(22) Filed: Feb. 26, 2024

Related U.S. Application Data
(60) Provisional application No. 63/448,633, filed on Feb. 27, 2023.

(51) Int. Cl. A61K 31/343; A61K 9/00; A61K 9/127; A61K 31/04; A61K 31/4985; A61K 47/44; A61P 27/02

(52) U.S. Cl. CPC A61K 31/343; A61K 9/0019; A61K 9/127; A61K 31/04; A61K 31/4985; A61K 47/44; A61P 27/02

(57) ABSTRACT

The present disclosure relates to pharmaceutical compositions and methods of their use for treating ocular diseases. Effective pharmaceutical compositions comprise a nitric oxide releasing agent. Also disclosed herein is an ophthalmic composition comprising a nitric oxide releasing agent configured for suprachoroidal injection, and a method of suprachoroidal injection administration thereof, to treat choroidal vascular diseases.

### FIELD

This disclosure relates to the field of ophthalmological compositions and methods of their use for treating ocular choroidal vascular diseases. In particular, this disclosure relates to a composition comprising a nitric oxide releasing agent, and methods of their administration to treat vascular diseases. The vascular diseases which can be treated include choroidal vascular diseases including central serous chorioretinopathy (CSCR), diabetic choroidopathy, hypertensive choroidopathy, choroidal ischemia, geographic macular atrophy as in non-exudative age-related macular degeneration and choroidal neovascularization as in age-related macular degeneration.

### RELATED APPLICATIONS

This application claims priority to U.S. Provisional Application No. 63/448,633, filed Feb. 27, 2023, the contents of which are herein incorporated in its entirety.

### BACKGROUND

The following includes information that may be useful in understanding the present invention. It is not an admission that any of the information, publications or documents specifically or implicitly referenced herein is prior art, or essential, to the presently described or claimed inventions. All publications and patents mentioned herein are hereby incorporated by reference in their entirety.

Nitric oxide (NO) is a potent neuro-transmitter and vasodilator. Parasympathetic innervations of the choroid release NO and VIP (Vasoactive intestinal peptide) as neuro-transmitters to regulate the choroidal and retinal blood flow by causing vasodilation by relaxation of vascular smooth muscles.

The sclera and RPE is innervated by parasympathetic nervous system with short ciliary nerves penetrating the sclera near the optic nerve region at the posterior aspect of the eyeball. While many of the parasympathetic nerves in the eye use muscarinic acetylcholine as the main neurotransmitter, i.e. those that supply the ciliary body and iris, some of the parasympathetic nerves that innervate and regulate the choroidal vasculature are innervated by neurons that use nitric oxide (NO) and vasoactive intestinal peptide (VIP).

Central serous chorioretinopathy (CSCR) is the fourth most common retinopathy (Wang M et al. Central serous chorioretinopathy. Acta Ophthalmo (Copenn) 2008; 86 (2) 126-145). No underlying pathophysiologic mechanism has been proven, but CSCR is thought to occur due to hyper-permeable choroidal capillaries, causing a serous detachment of the neurosensory retina. Risk factors for CSCR include smoking, steroid use, hyperopia and type A personality. These risk associations yield important clues about the pathogenesis of CSCR, and suggest the parasympathetic is under-active. The common element for these risk factors can be viewed as nitric oxide deficiency in the choroidal vessels, leading to vasoconstriction and flow backup, which leads to vascular leakage and sub-retinal fluid accumulation.

Smoking and steroid use are both known to decrease the nitric oxide (NO) production. Studies have shown the corticosteroids have an inhibitory effect on NO production (Linehan J D et al. Effect of corticosteroids on nitric oxide production in inflammatory bowel disease: are leukocytes the site of action? Am J Physio Gastrointest Liver Physiol 2005 February; 288(2): G261-7). Pregnancy, which is also associated with CSCR, may lead to over-production of serum cortisol, a form of steroid. Myopia has been shown to be protective for CSCR in multiple studies (Manayath G J et al. Is myopia a protective factor against central serous chorioretinopathy? Int J Ophthalmol. 2016; 9(2): 266-270). Myopia and Type A personality can be viewed together because they both point to an imbalance between sympathetic and parasympathetic stimulation by the ciliary ganglion, which innervates the posterior pole choroidal vessels. Myopia development is likely driven by parasympathetic neuronal stimulation. During the development of myopia, when focusing on objects up close, accommodation is mainly governed by parasympathetic nerves which causes the ciliary muscle to contract and pupils to constrict, bringing the lens to a greater convexity and allowing the eye to focus on a near target. At the same time, the same parasympathetic nerves, which originate from the ciliary ganglion, also provide parasympathetic signals to the RPE and sclera in the posterior pole, causing fibroblasts to activate and cause scleral remodeling and axial elongation of the eye.

All of the previous concepts in treating ocular diseases have not yielded a fully effective treatment for central serous chorioretinopathy. There is also no local application of a drug that increases the choroidal blood flow. In particular, nitric oxide-releasing agents have not been administered by topical, subtenon or suprachoroidal administration to the eye as a clinical therapy to treat choroidal vascular diseases which can include or exclude central serous chorioretinopathy, diabetic choroidopathy, hypertensive choroidopathy, choroidal ischemia, geographic atrophy from non-exudative age-related macular degeneration (Dry ARMD) and choroidal neovascularization such as in exudate age-related macular degeneration (Wet ARMD).

### SUMMARY

The inventions described and claimed herein have many attributes and aspects including, but not limited to, those set forth or described or referenced in this Brief Summary. It is not intended to be all-inclusive and the inventions described and claimed herein are not limited to or by the features or embodiments identified in this Brief Summary, which is included for purposes of illustration only and not restriction.

This disclosure is directed to the field of vasodilation of the choroidal blood vessels. In particular, treatments are provided for ocular choroidal conditions which can include or exclude central serous chorioretinopathy, diabetic choroidopathy, hypertensive choroidopathy, choroidal ischemia, geographic atrophy from non-exudative age-related macular degeneration (Dry ARMD) and choroidal neovascularization such as in exudate age-related macular degeneration (Wet ARMD) where there is an interruption or reduction of choroidal blood flow. There remains along-felt but unmet need in the art for effective treatment of CSCR with reduced side effects of systemic nitric-oxide releasing compounds such as nitroglycerin. The present invention fulfills these and other needs.

In one aspect, this disclosure provides for a method for treating an ocular choroidal vascular disease in a subject in need thereof, said method comprising administering to the eye of a subject in need thereof a therapeutically effective amount of a dosage of a composition comprising a nitric oxide-releasing agent. In some aspects, the ocular vascular disease can be of the choroid. In some aspects, the administering to the subject can comprise contacting the sclera or the conjunctiva of the subject with said nitric oxide-releasing agent. In some aspects, the contacting the sclera or the conjunctiva can be performed by subtenon injection to the eye of said subject. In some aspects, the administering to the subject can comprise contacting the bulbar conjunctiva of the eye of said subject. In some aspects, the contacting the sclera or the conjunctiva can be performed by topical application to the eye of said subject. In some aspects, the contacting the sclera or the conjunctiva can be performed by suprachoroidal injection to the eye of said subject. In some aspects, the ocular vascular disease can be selected from central serous chorioretinopathy (CSCR), diabetic choroidopathy, hypertensive choroidopathy, choroidal ischemia, or geographic macular atrophy from dry ARMD and choroidal neovascularization as in wet ARMD. In some aspects, the ocular vascular disease is central serous chorioretinopathy.

In some aspects, the nitric oxide-releasing agent can be selected from isosorbide dinitrate, isosorbide mononitrate, nitroglycerin (1,2,3-Tris(nitrooxy)propane), or a combination thereof. In some aspects, the nitric oxide-releasing agent can be the combination of nitroglycerin and isosorbide dinitrate. In some aspects, the nitric oxide-releasing agent can be the combination of nitroglycerin and isosorbide mononitrate. In some aspects, the nitric oxide-releasing agent can be the combination of isosorbide dinitrate and isosorbide mononitrate.

In some aspects, the method can further comprise administering to the subject a therapeutically effective amount of a phosphodiesterase 5 inhibitor. The phosphodiesterase 5 inhibitor can be selected from sildenafil (Viagra), tadalafil (Cialis), and vardenafil (Levitra), avanafil (Stendra), combinations thereof, and pharmaceutically acceptable salts thereof.

In some aspects, the dosage of the composition can comprise 0.05-4% by weight, based upon the total weight of said composition, of said nitric oxide-releasing agent. In some aspects, the composition can include or exclude an effective amount of an anesthetic. The anesthetic can be present in an amount from about 0.1% to about 10% by weight, based upon the total weight of said composition. In some aspects, the anesthetic is a topical anesthetic. The topical anesthetic can be selected from proparacaine, dibucaine, pramoxine, prilocaine, dibucaine, benzocaine, tetracaine lidocaine, tetracaine, rodocaine (N-(2-chloro-6-methylphenyl)octahydro-trans-1H-pyrindine-1-propanamide), bupivacaine, benoxinate, and combinations thereof. In some aspects, the composition can include or exclude propylene glycol and a non-ionic surfactant. In some aspects, the non-ionic surfactant can be a member selected from the group consisting of sorbitan sesquioleate, sorbitan monostearate, propylene glycol monolaurate, sorbitan mono-oleate, glycerol monostearate, propylene glycol monostearate, sorbitan tristearate, and sorbitan trioleate. In some aspects, the non-ionic surfactant can be sorbitan sesquioleate. In some aspects, the composition can comprise an ionic surfactant. In some aspects, the ionic surfactant is Benzalkonium Chloride.

In some aspects, this disclosure provides for a composition which can be adapted for suprachoroidal injection to the suprachoroidal space (SCS) of a subject. In some aspects, the composition adapted for suprachoroidal injection comprises a liposome, wherein the nitric oxide-releasing agent is configured to substantially be within the liposome.

In some aspects, this disclosure provides for a composition which can be adapted for topical application over the conjunctiva or sclera of the eye. In some aspects, the composition can be adapted for subtenon injection to the eye. In some aspects, the composition can be adapted for suprachoroidal injection to the eye. In some aspects, the composition can include or exclude one or more pharmaceutically acceptable carriers or excipients in an admixture with said nitric oxide-releasing agent. In some aspects, the nitric oxide-releasing agent can be selected from isosorbide dinitrate, isosorbide mononitrate, nitroglycerin (1,2,3-Tris(nitrooxy)propane), or combinations thereof. In some aspects, the composition can be a combination of a plurality of nitric oxide-releasing agents described herein. In some aspects, the composition can include or exclude excipients which can include or exclude: white petrolatum, canola oil, mineral oil, lanolin, parrafin wax, distilled water, acetone sodium bisulfite, zinc oxide, cocoa butter, or mixtures thereof. In some aspects, the composition can be an injectable fluid. In some aspects, the composition can comprise: 0.05-4% percent by weight nitroglycerin, propylene glycol, and a non-ionic surfactant. In some aspects, the non-ionic surfactant is a member selected from the group consisting of sorbitan sesquioleate, sorbitan monostearate, propylene glycol monolaurate, sorbitan mono-oleate, glycerol monostearate, propylene glycol monostearate, sorbitan tristearate, and sorbitan trioleate. In some aspects, the non-ionic surfactant is sorbitan sesquioleate.

In some aspects, this disclosure provides for a method for treating ocular choroidal vascular disease in a subject in need thereof, wherein said composition can be administered to the eye at least one time daily. In some aspects, the composition can be administered 2-8 times daily.

In some aspects, this disclosure provides for a kit comprising: a composition comprising 0.05-4% percent by weight nitroglycerin, a container, directions for use, a measuring device, and optionally an applicator.

This description also discloses methods of designing and manufacturing such compositions and/or kits.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows a Wide-field color fundus photography image of the choroid at baseline.

FIG. 2 shows a Wide-field color fundus photography image of the choroid in a subject after 10 min post-injection of a representative nitric oxide releasing agent of this disclosure.

FIG. 3 shows a Wide-field color fundus photography image of the choroid in a subject after 20 min post-injection of a representative nitric oxide releasing agent of this disclosure.

FIG. 4 shows a Wide-field color fundus photography image of the choroid in a subject after 30 min post-injection of a representative nitric oxide releasing agent of this disclosure.

### DETAILED DESCRIPTION

### Definitions

As used herein, the recitation of a numerical range for a variable is intended to convey that the invention may be practiced with the variable equal to any of the values within that range. Thus, for a variable that is inherently discrete, the variable can be equal to any integer value of the numerical range, including the end-points of the range. Similarly, for a variable, which is inherently continuous, the variable can be equal to any real value of the numerical range, including the end-points of the range. As an example, a variable which is described as having values between 0 and 2, can be 0, 1 or 2 for variables which are inherently discrete, and can be 0.0, 0.1, 0.01, 0.001, or any other real value, for variables which are inherently continuous.

As used herein, the term “nitric oxide releasing agent” or “NO-releasing agent” refers to compounds and biomolecules which release nitric oxide into the selected target area in the body. In some embodiments, the NO-releasing agent is selected from: Nitroglycerin (Glyceryl trinitrate) (1,2,3-Trinitroxypropane), Isosorbide Dinitrate (ISDN) (1,4:3,6-dianhydro-2,5-di-O-nitro-D-glucitol), Isosorbide Mononitrate (ISMN) (8-nitrooxy-2,6-dioxabicyclo[3.3.0]octan-4-ol), Amyl Nitrate, Pentyl nitrate, Pentaerythritol tetranitrate (PETN) (2,2-Bis[(nitrooxy)methyl]propane-1,3-diyl dinitrate), Sodium nitroprusside (also referred to in some embodiments as: Sodium pentacyanidonitrosylferrate(III), or sodium pentacyanonitrosylferrate (iii) dihydrate-disodium), S-nitroso-N-acetylcystein, S-nitroso-acetylpenicillamine, spermine-NO, V-PYRRO-NO, diethylamine-NO, sodium pentacyanidonitrosylferrate(II), Nicorandil (also referred to in some embodiments as 2-Nicotinamidoethyl nitrate, Nipradilol (8-[2-hydroxy-3-(isopropylamino)propoxy]-3-chromanol,3-nitrate), N-(2-Hydroxyethyl)nicotinamide nitrate, N-(2-Hydroxyethyl)nicotinamide nitrate (ester), N-(2-Hydroxyethyl)nicotinamide nitrate), gluceryl trinitrate (GTN) (1,2,3-Trinitroxypropane), 2MNS-6 (Butyl 3,6-anhydro-2-O-nitro-b-D-glucofuranoside), DNS-1 (Butyl 3,6-anhydro-2,5-di-O-nitro-a-D-arabinohexofuranoside), DNS-2 (Butyl 3,6-anhydro-2,5-di-O-nitro-b-D-arabinohexofuranoside), 2MNS-8 (Butyl 3,6-anhydro-5-O-(4-methoxybenzyl)-2-O-nitro-b-D-glucofuranoside), Nitroaspirin, also referred to in some embodiments as NCX 4016 (2-(Acetyloxy)-benzoic acid 3-[(nitrooxy)methyl]phenyl ester), Diethylene glycol dinitrate—Nitric acid (2-(2-nitrooxyethoxy)ethyl ester), Itramin tosylate (Itramin tosylate) (2-aminoethyl nitrate; 4-methylbenzene-1-sulfonic acid), Propatylnitrate (2,2-bis(nitrooxymethyl)butyl nitrate), Sinitrodil (2-(4-Oxo-2H-1,3-benzoxazin-3(4H)-yl)ethyl nitrate), Tenitramine (2-[2-[bis(2-nitrooxyethyl)amino]ethyl-(2-nitrooxyethyl)amino]ethyl nitrate), Trolnitrate (2-[bis(2-nitrooxyethyl)amino]ethyl nitrate), Linsidomine (3-morpholinosydnonimine or SIN-1) (3-morpholin-4-yl-1-oxa-3-azonia-2-azanidacyclopent-3-en-5-imine), Diethylenetriamine/nitric oxide adduct (DETA-NO) (1-(2-Aminoethyl)-1-(2-ammonioethyl)-3-oxo-2-triazanolate), Mannitol hexanitrite, Mannitol hexanitrate ([(2R,3R,4R,5R)-1,2,4,5,6-Pentanitrooxyhexan-3-yl] nitrate), Inositol hexanitrite, and Inositol hexanitrate (1,2,3,4,5,6-Cyclohexanehexayl hexanitrate). In some embodiments, the NO-releasing agent can include or exclude 2-aryl-β-thiophens, described for example, in U.S. Pat. No. 5,811,437, incorporated herein by reference or other compounds described, for example, in U.S. Pat. No. 5,478,946, incorporated herein by reference. In some embodiments, the NO-releasing agent can include or exclude nitrosated and nitrosylated phosphodiesterase inhibitor compounds, including those described in U.S. Pat. Nos. 5,958,926, and 5,874,437, each of which are herein incorporated by in their entirety. In some embodiments, the NO-releasing agent can include or exclude: S-nitrosothiols (S-nitroso-L-cysteine, S-nitroso-L-homocysteine, S-nitroso-glutathione (GSNO), S-nitrosoalbumin, S-nitrosocaptopril, SNAP(D)-Gly-O-n-Bu), O-nitrite compounds, N-nitroso compounds (N-methyl-N-nitrosoaniline, dephostatin, diazeniumdiolates, N-hydroxy-N-nitrosoamines (cupferron), N-nitrosoureas), C-nitroso compounds (FK409), heterocyclic NO-donors (molsidomine, prazosin, oxadiazoles, oxatriazoles), transition metal NO-complexes (Roussin's red salt), SNO-moxisylyte (NMI-221), NCX-4215, S-nitroso-diclofenac, SNO-yohimbine (NMI-187), S-nitroso-N-acetylpenicillamine (SNAP), NCX 4016 (nitroaspirin), NO-fluvastatin (NCX-6553), and NO-pravastain (NCX 6550). In some embodiments, the NO-releasing agent can include or exclude S-Nitroso-NSAIDS (e.g., Naproxcinod).

In some embodiments, the NO-releasing agent is selected from: Nitroglycerin (Glyceryl trinitrate) (1,2,3-Trinitroxypropane), Isosorbide Dinitrate (1,4:3,6-dianhydro-2,5-di-O-nitro-D-glucitol), Isosorbide Mononitrate (8-nitrooxy-2,6-dioxabicyclo[3.3.0]octan-4-ol), Amyl Nitrate, Pentyl nitrate, Pentaerythritol tetranitrate (PETN) (2,2-Bis[(nitrooxy)methyl]propane-1,3-diyl dinitrate), Sodium nitroprusside (also referred to in some embodiments as: Sodium pentacyanidonitrosylferrate(III), sodium pentacyanonitrosylferrate (III) dihydrate-disodium), S-nitroso-N-acetylcystein, S-nitroso-acetylpenicillamine, spermine-NO, diethylamine-NO, sodium pentacyanidonitrosylferrate(II), Nicorandil (also referred to as 2-Nicotinamidoethyl nitrate, Nipradilol (8-[2-hydroxy-3-(isopropylamino)propoxy]-3-chromanol,3-nitrate), N-(2-Hydroxyethyl)nicotinamide nitrate, N-(2-Hydroxyethyl)nicotinamide nitrate (ester), N-(2-Hydroxyethyl)nicotinamide nitrate), gluceryl trinitrate (1,2,3-Trinitroxypropane), Butyl 3,6-anhydro-2-O-nitro-b-D-glucofuranoside, Butyl 3,6-anhydro-2,5-di-O-nitro-a-D-arabinohexofuranoside, Butyl 3,6-anhydro-2,5-di-O-nitro-b-D-arabinohexofuranoside, Butyl 3,6-anhydro-5-O-(4-methoxybenzyl)-2-O-nitro-b-D-glucofuranoside, Nitroaspirin, (2-(Acetyloxy)-benzoic acid 3-[(nitrooxy)methyl]phenyl ester), Diethylene glycol dinitrate—Nitric acid (2-(2-nitrooxyethoxy)ethyl ester), Itramin tosylate (Itramin tosylate) (2-aminoethyl nitrate; 4-methylbenzene-1-sulfonic acid), Propatylnitrate (2,2-bis(nitrooxymethyl)butyl nitrate), Sinitrodil (2-(4-Oxo-2H-1,3-benzoxazin-3(4H)-yl)ethyl nitrate), Tenitramine (2-[2-[bis(2-nitrooxyethyl)amino]ethyl-(2-nitrooxyethyl)amino]ethyl nitrate), Trolnitrate (2-[bis(2-nitrooxyethyl)amino]ethyl nitrate), Linsidomine (3-morpholinosydnonimine) (3-morpholin-4-yl-1-oxa-3-azonia-2-azanidacyclopent-3-en-5-imine), Diethylenetriamine/nitric oxide adduct (1-(2-Aminoethyl)-1-(2-ammonioethyl)-3-oxo-2-triazanolate), Mannitol hexanitrite, Mannitol hexanitrate ([(2R,3R,4R,5R)-1,2,4,5,6-Pentanitrooxyhexan-3-yl] nitrate), Inositol hexanitrite, and Inositol hexanitrate (1,2,3,4,5,6-Cyclohexanehexayl hexanitrate).

In some embodiments, the NO-releasing agent can include or exclude 2-aryl-β-thiophens, described for example, in U.S. Pat. No. 5,811,437, incorporated herein by reference or other compounds described, for example, in U.S. Pat. No. 5,478,946, incorporated herein by reference. In some embodiments, the NO-releasing agent can include or exclude nitrosated and nitrosylated phosphodiesterase inhibitor compounds, including those described in U.S. Pat. Nos. 5,958,926, and 5,874,437, each of which are herein incorporated by in their entirety.

In some embodiments, the NO-releasing agent can include or exclude: S-nitrosothiols (S-nitroso-L-cysteine, S-nitroso-L-homocysteine, S-nitroso-glutathione (GSNO), S-nitrosoalbumin, S-nitrosocaptopril, SNAP(D)-Gly-O-n-Bu), O-nitrite compounds, N-nitroso compounds (N-methyl-N-nitrosoaniline, dephostatin, diazeniumdiolates, N-hydroxy-N-nitrosoamines (cupferron), N-nitrosoureas), C-nitroso compounds (FK409), heterocyclic NO-donors (molsidomine, prazosin, oxadiazoles, oxatriazoles), transition metal NO-complexes (Roussin's red salt), SNO-moxisylyte (NMI-221), NCX-4215, S-nitroso-diclofenac, SNO-yohimbine (NMI-187), S-nitroso-N-acetylpenicillamine (SNAP), NCX 4016 (nitroaspirin), NO-fluvastatin (NCX-6553), and NO-pravastain (NCX 6550). In some embodiments, the NO-releasing agent can include or exclude S-Nitroso-NSAIDS (e.g., Naproxcinod). In some embodiments, the nitric oxide releasing agent can include or exclude sodium nitroprusside or diazenium diolates. In some embodiments, the nitric oxide-releasing agent is a “NONOates.” NONOates can include or exclude (Z)-1-{N-methyl-N-[6-(N-methyl-ammoniohexyl)amino]} diazen-1-ium-1,2-diolate (“MAHMAINO”), (Z)-1-[N-(3-ammoniopropyl)-N-(n-propyl)amino]-diazen-1-ium-1,2-diolate (“PAPA/NO”), (Z)-1-{N-[3-aminopropyl]-N-[4-(3-aminopropylammonio)butyl]amino} diazen-1-ium-1,2-diolate (spermine NONOate or “SPER/NO”) and sodium (Z)-1-(N,N-diethylamino)-diazen-1-ium-1,2-diolate (diethylamine NONOate or “DEA/NO”) and derivatives thereof). In some embodiments, the nitric oxide releasing agent is selected from: nitroglycerin, isosorbide dinitrate, erythrityl tetranitrate, amyl nitrate, molsidomine, linsidomine chlorhydrate (“SIN-1”), S-nitroso-N-acetyl-d,l-penicillamine (“SNAP”) and S-nitroso-N-glutathione (“SNO-GLU”).

As used herein, the term “subject” or “subject in need thereof” refers to humans as well as non-human animals, such as domesticated mammals including, without limitation, cats, dogs, rabbits, and horses. The formulations and methods of the present invention are intended for use with any subject that may experience the benefits of the formulations and methods of the invention. The subject is typically a mammal, more typically a human. However, the invention is not limited to the treatment of humans and is applicable to veterinary uses.

As used herein, the term “therapeutically effective amount” refers to treatments at dosages effective to achieve the therapeutic result sought.

The term “therapeutically effective amount” or “effective amount” means the amount of the subject compound that will elicit a desired response, for example, a biological or medical result or response of a tissue, system, animal or human that is sought, for example, by a researcher, veterinarian, medical doctor, or other clinician. That result can be alleviation of the signs, symptoms, or causes of a disease or disorder or condition, or any other desired alteration of a biological system. In the present invention, the result will typically involve, for example, the prevention, decrease, or reversal of tissue injury or damage, in whole or in part, associated with central serous chorioretinopathy (CSCR), diabetic choroidopathy, hypertensive choroidopathy, choroidal ischemia, geographic atrophy from dry ARMD and choroidal neovascularization from wet ARMD.

As used herein, “treatment” refers to clinical intervention in an attempt to alter the natural course of the individual or cell being treated, and can be performed either for prophylaxis or during the course of clinical pathology. Desirable effects of treatment include preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies of the invention are used to delay development of a disease or disorder. An “effective amount” refers to an amount effective, at dosages and for periods of time necessary, to achieve the desired therapeutic or prophylactic result. A “therapeutically effective amount” of a substance/molecule of the invention, agonist or antagonist may vary according to factors such as the disease state, age, sex, and weight of the individual, and the ability of the substance/molecule, agonist or antagonist to elicit a desired response in the individual. A therapeutically effective amount is also one in which any toxic or detrimental effects of the substance/molecule, agonist or antagonist are outweighed by the therapeutically beneficial effects. A “prophylactically effective amount” refers to an amount effective, at dosages and for periods of time necessary, to achieve the desired prophylactic result. Typically but not necessarily, since a prophylactic dose is used in subjects prior to or at an earlier stage of disease, the prophylactically effective amount will be less than the therapeutically effective amount.

As used herein, the term “composition” refers to a product comprising one or more ingredients.

Treating Choroidal Vascular Diseases with Nitric Oxide-Releasing Agents

Nitric oxide-releasing agents described herein can be used to treat choroidal vascular diseases. Nitric oxide (NO) is a potent neuro-transmitter and vasodilator. Parasympathetic innervations of the choroid release NO and VIP (Vasoactive intestinal polypeptide) as neurotransmitters to regulate the choroidal blood flow by causing vasodilation.

Myopia development is likely driven by parasympathetic neuronal stimulation. During the development of myopia, when focusing on objects up close, accommodation is mainly governed by parasympathetic nerves which causes the ciliary muscle to contract and pupils to constrict, bringing the lens to a greater convexity and allowing the eye to focus at a near object. At the same time, the same parasympathetic nerves, which originate from the ciliary ganglion, also provide parasympathetic signals to the RPE and sclera in the posterior pole, causing fibroblasts to active and causing scleral remodeling and axial elongation of the eye. In myopic patients, this parasympathetic stimulation overwhelms the sympathetic stimulation from the ciliary ganglion, which protects against CSCR as NO release is mainly governed by parasympathetic neurons. Type A personality will also provide over-activity of the sympathetic nerve system, which again works against the parasympathetic nervous system and decreases NO production. Thus, CSCR is caused by decreased production or bioavailability of NO, leading to vascular congestion and accumulation of plasma/fluid in the subretinal space.

In some embodiments, this disclosure provides for nitric oxide-releasing agents which can cause choroidal vessel vasodilation so as to treat choroidal vascular diseases which can include or exclude central serous chorioretinopathy, diabetic choroidopathy, hypertensive choroidopathy, choroidal ischemia, geographic atrophy from ARMD and choroidal neovascularization from wet ARMD. Also described herein are methods of increasing the bioavailability of NO to the choroidal vasculature, thereby leading to vasodilation and decongestion of the choroidal vasculature.

One embodiment of the present invention takes advantage of the fact that the choroidal and retinal vasculature are significantly different. There are two distinct vascular systems which support the adult neural retina—the proper retinal vessels and the choroidal vessels. The two blood vessel types differ in their embryonic differentiation, adult organization, and cellular composition. The retinal vasculature has barrier properties similar to those observed in the brain, whereas the choroidal vessels display a highly fenestrated phenotype and are thus “leaky” in nature. (Saint-Geniez, M., et al., Development and pathology of the hyaloid, choroidal and retinal vasculature. Int J Dev Biol. 2004; 48(8-9):1045-58). The retina's blood supply dependency makes it highly vulnerable to any vascular changes from variousocular diseases, which are associated with abnormalities of the different vascular beds of the eye. Retinal blood vessels are organized in two planar layers that are restricted to the inner layers of the retina. As in the central nervous system, the retinal vascular network is characterized also by its blood-barrier status. This specialization of the endothelial cells is apparently induced by interaction with other cells that participate in retinal vascularization including the Müller cells (Tout, S., et al. (1993), The role of Muller cells in the formation of the blood-retinal barrier. Neuroscience 55: 291-301) and the astrocytes (Janzer, et al., (1987). Astrocytes induce blood-brain barrier properties in endothelial cells. Nature 325: 253-257). Retinal vessels are present within the central nervous system (CNS), and are vascularized through the angiogenic invasion of blood vessels from a perineural vascular plexus, followed by continued sprouting and remodeling until a hierarchical vascular network is formed. Retinal vessels, however, are not innervated, and thus do not respond to NO as a neurotransmitter. For this reason, nitric oxide-releasing agents, including nitroglycerin, are not highly efficacious in ocular diseases targeting the retina and/or the optic nerve.

The choroid is a highly vascularized tissue layer that lies outside of the blood brain barrier. More specifically, it lies outside of the blood retina barrier formed by the tight junctions of the retinal pigment epithelium layer (RPE). A choroid-targeted drug would be presented to the choroid by direct penetration without having to cross the blood retina barrier by local administration of a nitric oxide-releasing agent described herein. Choroid-targeted drugs only need to pass through the sclera to access the choroid. Treatments for glaucoma and central retinal artery occlusion, for example, are targeted to a different type of circulation system, comprising different blood vessels than the choroid. The choroid and retinal vasculature are physiologically different. For example, the choroid comprises different wall thicknesses (e.g., capillaries), and is very leaky in nature, and thus exhibit different drug transport profiles than inner retinal blood vessels which are less penetratable to drugs due to presence of gap junctions. Moreover, choroidal and retinal blood vessels are innervated differently, such that signaling for one blood vessel type may not necessarily work in the other blood vessel type.

In some embodiments, the nitric-oxide releasing agent can be administered by suprachoroidal injection the eye. Suprachoroidal injection affords presentation of a drug to the choroid, retinal pigment epithelium and retina with high bioavailability, while maintaining low levels elsewhere in the eye (Chiang et al., The suprachoroidal space as a route of administration to the posterior segment of the eye, Adv Drug Deliv Rev. 2018 Feb. 15; 126: 58-66. doi: 10.1016/j.addr.2018.03.001; PMID: 29545195),

Aqueous humor outflow allows for fluid in the eye to escape by a variety of mechanisms, reducing intra-ocular pressure. Thus, the IOP lowering effect of nitroglycerin is not expected to be useful for treating choroidal diseases, including central serous chorioretinopathy.

Presently, there are no drug-based treatments specific to choroidal diseases including central serous chorioretinopathy, diabetic or hypertensive retinopathy, choroidal ischemia and choroidal neovascularization. While nitroglycerin has been attempted for some types of ocular diseases, it has been met with mixed results depending on the supporting vasculature and etiology of the disease. CSCR has been regarded as a disease caused by vasodilation as demonstrated by leakage on fluorescein angiography, and generally not recognized as a disease from vasoconstriction. In one embodiment, the choroidal vasculature is locally targeted to achieve vasodilation to counter the effects of vasoconstriction in CSCR using a nitric oxide-releasing agent described herein.

The unique anatomy and physiology of the eye has made ocular drug delivery a major challenge to pharmacologists. The combination of static barriers (different layers of cornea, sclera, and retina including blood aqueous and blood-retinal barriers), dynamic barriers (choroidal and conjunctival blood flow, lymphatic clearance, and tear dilution), and efflux pumps pose a significant challenge for delivery of a drug alone or in a dosage form, especially to the posterior segment. In some embodiments, this invention provides for an ophthalmologically safe and effective dosage and a route of administration to the posterior sclera to treat choroidal vascular diseases which can include or exclude central serous chorioretinopathy, diabetic or hypertensive choroidopathy, choroidal ischemia and choroidal neovascularizationby local administration of a nitric oxide-releasing agent. In some embodiments, the route of administration is the application of an ophthalmic ointment on the conjunctiva in the superior fornix at the superotemporal and/or in the inferior fornix at the inferotemporal quadrant. In some embodiments, the route of administration is suprachoroidal injection of the nitric oxide-releasing agent to the eye of a subject. In some embodiments, the route of administration is subtenon injection of the nitric oxide-releasing agent to the eye of a subject.

Nitric oxide has been shown to be a key neuro-transmitter in causing vasodilation in multiple studies. (Nilsson S., et al. Exper Eye Res, 70(1), 61-72 January 2000). NO has also been shown to inhibit sympathetic nervous system in the brain. (Kishi T., Regulation of the sympathetic nervous system by nitric oxide. Hypertension research 36, 845-851(2013)). Local administration of a nitric oxide releasing agent, which can lead to a direct effect on the choroidal vessels with less undesirable systemic effects such as headaches, hypotension and tachycardia, is a preferred embodiment for treating eye conditions. Maintaining local administration of the nitric oxide-releasing agent following suprachoroidal administration to the eye of a subject can be performed using the compositions formulated for suprachoroidal administration as described herein.

In some embodiments, the nitric oxide-releasing agent is nitroglycerin. Nitroglycerin is a medication which can be systemically administered orally, transdermally and transmucosally to treat angina, heart failure and anal fissures. Nitroglycerin ointment has been tested on external ocular structures in the anterior chamber of rabbit eyes and has demonstrated overall safety except for allergic type reactions, which is likely dose-mediated (Zalta A et al. Journal of Ocular Pharmacology. Vol 1, Number 1, 1985.) Oral nitroglycerin has also been shown to have beneficial effects for glaucoma patients and diabetics, where the retinal blood flow improves after taking nitroglycerin orally (Zurakowski D et al. Nitrate therapy may retard glaucomatous optic neuropathy, perhaps through modulation of cyclic GMP pathway on vasodilation of vascular smooth muscles. Vision Research 38 (1998) 1489-1494; Weigert G et al. Nitroglycerin-Mediated Retinal Vasodilatation Is Maintained In Patients With Diabetes. IOVS May 2008 Vol 49, 2088). Nitroglycerin has been used as sublingual route to treat acute central retinal artery occlusion with variable success. Nitroglycerin has not been used in subtenon injection for localized targeted treatment of choroidal vascular diseases including CSCR, diabetic choroidopathy, hypertensive choroidopathy, choroidal ischemia and/or choroidal neovascularization. In one embodiment, this disclosure provides for an ophthalmically safe and effective dosage of a nitric oxide-releasing agent. In one embodiment, this disclosure provides for a method of treating ocular diseases by administering a composition comprising a nitric-oxide releasing agent to the posterior sclera by applying the composition to the conjunctiva in the superior fornix at the superotemporal quadrant.

In some embodiments, this disclosure provides for a method of preventing and/or treating damage and/or inflammation associated with choroidal vascular diseases which can include or exclude central serous chorioretinopathy (CSCR), diabetic choroidopathy, hypertensive choroidopathy, choroidal ischemia and choroidal neovascularization, wherein the damage and/or inflammation is prevented, ameliorated and/or delayed.

In some embodiments, this disclosure provides for a method of ameliorating tissue damage and/or enhancing tissue repair by locally administering one or more nitric oxide-releasing agents, to the site of tissue in need thereof. Such tissue may be damaged, or have been damaged, for example, as a result of a choroidal vascular disease which can include or exclude central serous chorioretinopathy (CSCR), diabetic or hypertensive choroidopathy, choroidal ischemia or choroidal neovascularization. Treatment may also be used to prevent and/or ameliorate and/or delay the onset of the choroidal vascular disease which can include or exclude central serous chorioretinopathy (CSCR), diabetic or hypertensive choroidopathy, choroidal ischemia or choroidal neovascularization by locally administering a nitric oxide-releasing agent to the choroidal vasculature.

In some embodiments the invention comprises a method of ameliorating tissue damage and/or enhancing tissue repair associated with a choroidal vascular disease which can include or exclude central serous chorioretinopathy (CSCR), diabetic or hypertensive choroidopathy, choroidal ischemia or choroidal neovascularization wherein the subject is a mammal. In another embodiment the invention comprises a method of ameliorating tissue damage and/or enhancing tissue repair associated with a choroidal vascular disease which can include or exclude central serous chorioretinopathy (CSCR), diabetic or hypertensive choroidopathy, choroidal ischemia or choroidal neovascularization, wherein the subject is human.

### Methods of Administration

Systemic administration of nitric oxide-releasing agents (e.g., nitroglycerin) involves unwanted side effects which may include toxicity. The side effects of systemic nitroglycerin can include or exclude headaches, hypotension (low blood pressure) and tachycardia (fast heart beat). Currently, there is no composition formulated for local application of nitric oxide-releasing agents to the eye, let alone to the choroidal vasculature.

In one embodiment, this disclosure provides for a method of improving nitric oxide-mediated vasodilation in the choroidal vasculature by locally administering a composition comprising a nitric oxide-releasing agent. In some embodiments, the nitric oxide-releasing agent is nitroglycerin. In some embodiments, the local administration is suprachoroidal injection. In some embodiments, the local administration is subtenon injection. In some embodiments, the local administration is topical application to the surface of the eye of a subject.

In some embodiments, this disclosure provides for a method of improving nitric oxide-mediated vasodilation in the choroidal vasculature by subtenon injection of a composition comprising a nitric oxide-releasing agent. The topically applied or subtenon injected nitric oxide-releasing agent penetrates through the conjunctiva and sclera to reach the choroidal vasculature to treat choroidal vascular diseases which can include or exclude central serous chorioretinopathy, diabetic and hypertensive choroidopathy, choroidal ischemia, dry ARMD, wet ARMD and choroidal neovascularization.

In one embodiment, the compositions described herein can be administered to the posterior sclera by applying the ophthalmic composition to the conjunctiva in the superior fornix at the superotemporal quadrant of the eye.

In some embodiments, the composition comprising the nitric oxide-releasing agent may be administered to the eye by injection, for example, by suprachoroidal injection, intraocular injection, intravitreal injection or by periocular routes including subconjunctival, retrobulbar, peribulbar, and posterior sub-tenon injections. In some aspects, the composition comprising the nitric oxide-releasing agent may be provided to or injected directly into or near the trabecular meshwork. The composition comprising the nitric oxide-releasing agent of this invention may also be administered at or near the trabecular meshwork, Episcleral vein, or ciliary body, so that the nitric oxide-releasing agent contacts the trabecular meshwork, Episcleral vein, and/or the ciliary body, respectively.

In some embodiments, the suprachoroidal injection for administering the nitric oxide-releasing agents to the SCS of the subject can be selected from suprachoroidal injection methods of ab interno surgical technique, by sclerotomy with subsequent micro-cannulation, or by microneedle injection to the SCS.

In some embodiments, the suprachoroidal injection can comprise sclerotomy (cutting across the sclera). In some embodiments, the sclerotomy can comprise micro-cannulation into the SCS (iScience catheter, Ellex Medical, Adelaide, Australia).

### Suprachoroidal Injections

In some embodiments, suprachoroidal injection of the nitric oxide-releasing agents of this disclosure to the SCS of a subject can comprise using a hypodermic needle to directly inject into the SCS. This can be a challenging procedure, since visualization of the scleral-choroidal plane is not possible with this method, and instead tactile cues can be used to indicate when the sclera has been penetrated. When used to evacuate suprachoroidal hemorrhage, there is more leeway in targeting an ideal depth because the SCS is expanded with blood. However, a high level of user experience is required when targeting the typically collapsed SCS using direct injection into the SCS. In direct injection, a 30 gauge hypodermic needle (˜300 μm outer diameter, bevel length >1 mm) is directed through the ˜500 μm thick sclera (Olsen T W, Aaberg S Y, Geroski D H, Edelhauser H F. Human sclera: thickness and surface area. Am J Ophthalmol. 1998; 125:237-241.) Direct injection may present safety issues from the high experience level required and thus variations between users.

In some embodiments, suprachoroidal injection can comprise the use of microneedles. Microneedles can deposit the nitric oxide-releasing agent into the SCS in a facile manner. A microneedle is a hollow-bore needle with a length matched to the thickness of the sclera and conjunctiva. The needle length is selected such that the microneedle is physically unable to penetrate deeper than the SCS (such as through the choroid and retina) and perform an inadvertent intravitreal injection. Fluid injected into this space spreads circumferentially within the SCS, bathing the choroid with the composition comprising the nitric oxide-releasing agent. A similar procedure used for intravitreal injection can be used with a microneedle for SCS injection, and can thus be performed in the outpatient clinic setting by ophthalmologists with patients under local anesthesia. In some embodiments, the microneedle is positioned perpendicular to the scleral surface and the hard stop at the hub at the microneedles' base contacts the sclera/conjunctiva to accurately control the insertion depth. The microneedle can be kept in position for ˜1 min to minimize reflux.

### Compositions

The compositions of the present disclosure are especially useful for treating individuals suffering from CSCR and other diseases with resulting choroidal ischemia. The compositions exhibit an improved safety profile resulting in significantly less headaches and hypotension. By locally applying the compositions described herein, other systemic toxicities associated with nitroglycerin can be reduced.

In some embodiments, this disclosure provides for a pharmaceutical composition comprising a nitric oxide-releasing agent, formulated for safe direct ocular application.

In some embodiments, the nitric oxide-releasing agents can be formulated as the direct injection of an aqueous solution to the SCS of the eye of a subject.

In some embodiments, the compositions comprising a nitric oxide-releasing agent can comprise nitroglycerin with lactose monohydrate (Copperhead Chemical, USA). In some embodiments, the compositions comprising a nitric oxide-releasing agent can comprise nitroglycerin with propylene glycol or ethanol (Copperhead Chemical, USA). In some embodiments, the compositions comprising a nitric oxide-releasing agent can comprise nitroglycerin in buffered saline.

In some embodiments, the composition comprising a nitric oxide-releasing agent is formulated for topical application. In some embodiments, the topical pharmaceutical composition is in the form of an ointment. In some embodiments, the ointment can comprise:
(a) about 0.05 to 4% by weight of nitric oxide-releasing agent,
(b) about 5 to 24% by weight of a penetration enhancer,
(c) about 60 to 90% by weight of water,
(d) about 0.5 to 3% by weight of a thickener, and
(e) about 0.4 to 2% by weight of an emulsifier.

In some embodiments, the compositions comprising a nitric oxide-releasing agent of this disclosure can include or exclude:
(f) about 0.5 to 4% ancillary ingredients, which can further include an anesthetic.

In some embodiments, the anesthetic can include or exclude: proparacaine, dibucaine, pramoxine, prilocaine, dibucaine, benzocaine, tetracaine lidocaine, tetracaine, rodocaine (N-(2-chloro-6-methylphenyl)octahydro-trans-1H-pyrindine-1-propanamide), bupivacaine, benoxinate, and combinations thereof. In some embodiments, the ocular anesthetic is a combination of lidocaine and bupivacaine.

In some embodiments, the compositions comprising nitric oxide-releasing agent of this disclosure can be formulated for suprachoroidal injection. Suprachoroidal injection is the direct injection of an active agent to the suprachoroidal space (SCS). The suprachoroidal space is a potential space between the sclera and choroid that traverses the circumference of the posterior segment of the eye. When compositions are formulated for suprachoroidal injection, they can be tailored to increase or maintain the location of the nitric oxide-releasing agent within the suprachoroidal space, such as by minimizing diffusion to other areas of the eye or minimizing clearance from the eye.

In some embodiments, the nitric-oxide releasing agent is substantially encapsulated within a liposome. The term “liposome” as used herein, refers to a particle characterized by an amphipathic spherical or near spherical bilayer formed by van der Waals interactions between a plurality of hydrophobic moieties each capped by a polar head group and arranged in an alternating manner such that a polar head group of one hydrophobic moiety projects outwards to an external aqueous environment, while an adjacent hydrophobic moiety projects its polar head group inward. Liposomes can be classified according to their lamellarity (uni- and multi-lamellar vesicles), size (small, intermediate, or large) and charge (anionic, cationic and neutral) of the polar head groups. In some embodiments, a liposome particle contains other hydrophobic components each of which is intercalated between two hydrophobic moieties of the liposome particle such that the optional hydrophobic components are embedded within the bilayer so as to affect the fluidity of the bilayer. In some embodiments, liposomes encapsulate a weakly charged or uncharged and/or poorly water soluble therapeutic agent by encapsulating that agent within its interior so as to separate that therapeutic agent from the external aqueous environment. Liposome particles typically have a diameter ranging from about 0.025 um to about 2.5 um in which the hydrophobic moieties are linear or lightly branched saturated hydrocarbons. In some aspects, a liposome particle is comprised of saturated phospholipids having acyl chains whose lengths are selected based upon the desired diameter of the liposome particle. In some embodiments, the phospholipid is selected from: Soybean phosphatidylcholine (SPC), Hydrogenated soybean phosphatidylcholine (HSPC), Egg sphingomyelin (ESM), Egg phosphatidylcholine (EPC), Dimyristoyl phosphatidylcholine (DMPC), Dipalmitoyl phosphatidylcholine (DPPC), Dioleoyl phosphatidylcholine (DOPC), Distearoyl phosphatidylcholine (DSPC), Dimyristoyl phosphatidylglycerol (DMPG), Dipalmitoyl phosphatidylglycerol (DPPG), Dioleoyl phosphatidylglycerol (DOPG), Distearoyl phosphatidylglycerol (DSPG), Dimyristoyl phosphatidylethanolamine (DMPE), Dipalmitoyl phosphatidylethanolamine (DPPE), Dioleoyl phosphatidylethanolamine (DOPE), Dimyristoyl phosphatidylserine (DMPS), Dipalmitoyl phosphatidylserine (DPPS), Dioleoyl phosphatidylserine (DOPS), 1,2-dioleoyl-3-trimethylammonium-propane (DOTAP), 1,2-dilauroyl-sn-glycero-3-phosphocholine (DLPC), 1,2-dimyristoyl-sn-glycero-3-phosphocholine (DMPC), 1,2-dimyristoyl-sn-glycero-3-phosphate (sodium salt) (DMPA.Na), 1,2-dipalmitoyl-sn-glycero-3-phosphate (sodium salt) (DPPA.Na), 1,2-dioleoyl-sn-glycero-3-phosphate (sodium salt) (DOPA.Na), 1,2-dimyristoyl-sn-glycero-3-phospho-(1′-rac-glycerol) (sodium salt) (DMPG.Na), 1,2-dipalmitoyl-sn-glycero-3-phospho-(1′-rac-glycerol) (sodium salt) (DPPG.Na), 1,2-dioleoyl-sn-glycero-3-phospho-(1′-rac-glycerol) (sodium salt) (DOPG.Na), 1,2-dimyristoyl-sn-glycero-3-phospho-L-serine (sodium salt) (DMPS.Na), 1,2-dipalmitoyl-sn-glycero-3-phospho-L-serine (sodium salt) (DPPS.Na), 1,2-dioleoyl-sn-glycero-3-phospho-L-serine (sodium salt) (DOPS.Na), 1,2-dioleoyl-sn-glycero-3-phosphoethanolamine-N-(glutaryl) (sodium salt) (DOPE-Glutaryl (Na)₂), 1′,3′-bis[1,2-dimyristoyl-sn-glycero-3-phospho]-sn-glycerol (ammonium salt) (Tetramyristoyl Cardiolipin (Na)₂), 1,2-distearoyl-sn-glycero-3-phosphoethanolamine-N-[methoxy(polyethylene glycol)-2000] (ammonium salt) (DSPE-mPEG-2000, Na), 1,2-distearoyl-sn-glycero-3-phosphoethanolamine-N-[methoxy(polyethylene glycol)-5000] (ammonium salt) (DSPE-mPEG-5000⋅Na), 1,2-distearoyl-sn-glycero-3-phosphoethanolamine-N-[maleimide(polyethylene glycol)-2000] (ammonium salt) (DSPE-Maleimide PEG-2000, Na), 1,2-dioleoyl-3-trimethylammonium-propane (chloride salt) (DOTAP, Cl), 1,2-dihexanoyl-sn-glycero-3-phosphocholine (DHPC), and mixtures thereof, and salts thereof. In some embodiments, the liposomes can be those described in S. Ansar et al., J Pharm Biomed Anal, 2022 Jan. 20; 208:114473. doi: 10.1016/j.jpba.2021.114473. Epub 2021 Nov. 18; Chang et al., Int J Nanomedicine. 2012; 7: 49-60; Published online 2011 Dec. 30. doi: 10.2147/IJN.S26766; or Ranjbar, et al., (2009). Acta Ophthalmologica, 87: 0-0.

In some embodiments, preparing liposomal compositions comprising nitric oxide-releasing agents of this disclosure can be performed using similar methods to prepare commercially available liposomal ocular pharmaceutical compositions. Such commercially available liposomal ocular pharmaceutical compositions include

Without being bound by theory, it is expected that the nitric oxide-releasing agent within the liposome maintains a high local concentration of the nitric oxide-releasing agent after suprachoroidal administration because the liposome reduces diffusion of the nitric oxide-releasing agent to areas of the eye outside of the suprachoroidal space. The high density of fibers and cells enclosing the suprachoroidal space minimize the diffusion of the relatively larger liposome particles more so than unliposomed nitric oxide-releasing agents. Thus, it is expected that compositions formulated for suprachoroidal injection comprising a nitric oxide-releasing agent encapsulated within a liposome will exhibit an extended drug-release profile. Such an extended drug-release profile can, in some embodiments, result in fewer administrations per unit time which is preferred by many patients for treatments involving injections to the eye.

In some embodiments, the compositions comprising a nitric oxide-releasing agent of this disclosure can include or exclude a phosphodiesterase 5 inhibitor (PDE5). While phosphodiesterase 5 inhibitors are usually contraindicated with nitric oxide-releasing agents, said contraindication is only for dual systemic administration of the PDE5 inhibitor and the nitric oxide-releasing agent. The inventor has recognized that local administration of a nitric oxide-releasing agent affords the systemic administration of a phosphodiesterase 5 inhibitor to achieve a synergistic effect on treating choroidal vascular diseases which can include or exclude central serous chorioretinopathy (CSCR). For example, the dosage of the nitric oxide-releasing agent can be changed (either increased or decreased, as appropriate) in concentration, number of administrations, or duration between administrations, when the subject to which the nitric oxide-releasing agent administered is also administered a PDE5 inhibitor, while achieving the same level of treatment. In some embodiments, the PDE5 inhibitor is selected from: sildenafil (Viagra), tadalafil (Cialis), avanafil (Stendra), and vardenafil (Levitra), combinations thereof, and pharmaceutically acceptable salts thereof. In some embodiments, the phosphodiesterase 5 (PDE5) inhibitors can be selected from those described in U.S. Pat. Nos. 6,037,346, 6,127,363, 6,156,753, 6,469,012, 6,300,335, and 6,548,490, each of which are herein incorporated by reference.

In some embodiments, the compositions comprising a nitric oxide-releasing agent of this disclosure comprising a nitric oxide-releasing agent can further comprise a non-ionic surfactant. The non-ionic surfactant can be selected from: sorbitan sesquioleate, sorbitan monostearate, propylene glycol monolaurate, sorbitan mono-oleate, glycerol monostearate, propylene glycol monostearate, sorbitan tristearate, and sorbitan trioleate. In some embodiments, the non-ionic surfactant can include or exclude polyoxyethylene alkyl ethers which can include or exclude the series of Spans, Tweens, and ester linked surfactants, Brij. The non-ionic surfactants can include or exclude: Brij 35, Brij 78, Brij 98, Brij 700, Polysorbate 20, Polysorbate 80, polyoxyl 40 stearate, polyoxyl 60, hydrogenated castor oil, Pluronic F127, Solulan C-24, Span 20, Span 40, Span 60, Span 80, Tween 20, Tween 40, Tween 80. In some embodiments, the emulsifier can include or exclude: lanolins, light mineral oil, mineral oil, paraffin, petrolatum, castor oil, and crosslinked polymers of acrylic acid (carbomer 1342).

In some embodiments, the compositions comprising a nitric oxide-releasing agent of this disclosure comprising a nitric oxide-releasing agent can further comprise a thickener. The thickener can include or exclude: methylparaben, propylparaben, ethyl cellulose, hydroxypropylmethyl cellulose, carboxymethyl cellulose, polyethylene glycol, isopropyl palmitate, polyoxyl stearate, or polyvinylalcohol.

In some embodiments, the compositions comprising a nitric oxide-releasing agent of this disclosure comprising a nitric oxide-releasing agent can further comprise a penetration enhancer selected from: bile salts, essential oils, terpenes, terpenoids. In some embodiments, the penetration enhancer can include or exclude: Sodium deoxycholate, Sodium taurodihydrofusidate, sodium taurocholate, Sodium glycodihydrofusidate, Cineole, eucalyptol, cyclodextrins described herein, dimethylsulphoxide (DMSO), ethylenediaminetetraacetic acid (EDTA), sodium glycocholate and related cholates, Tween and Brij surfactants described herein (including Tween 20, and Brij 35), saponins (including digitonin), sodium dodecyl sulfate, sodium octyl sulfate, sodium decyl sulfate, Azone® (1-dodecylazacycloheptan-2-one), ethylene glycol-bis(beta-aminoethyl)-N,N,N′,N′-tetraacetic acid (EGTA), 1,2-bis(o-aminophenoxy)ethane-N,N,N′,N′-tetraacetic acid (BAPTA), and ethylenediamine-N,N′-disuccinic acid (EDDS), and others which are well-known in the art. Penetration enhancers promote the penetration of drugs through the corneal barrier, and change the integrity of the epithelial cell layer.

In some embodiments, the compositions comprising a nitric oxide-releasing agent of this disclosure comprising a nitric oxide-releasing agent can further comprise ancillary ingredients selected from: chlorobutanol, hyaluronidase, epinephrine, buffering agents, and fluorescein. In some embodiments, the ancillary ingredients can include or exclude a preservative. Preservatives are well-known in the art and can include or exclude sodium perborate; Oxyd (sodium chlorite 0.05%, hydrogen peroxide 0.01%); polyquarternium-1 (ethanol, 2,2′,2″-nitrilotris-, polymer with 1,4-dichloro-2-butene and N,N,N′,N′-tetramethyl-2-butene-1,4-diamine); benzalkonium chloride; benzalkonium brome; sodium silver chloride; hexamethylene biguanide; oxyborate; and Purite® (sodium chlorite 0.005% m/v).

In some embodiments, the compositions of this disclosure comprising a nitric oxide-releasing agent does not include a preservative. Some types of preservatives can cause scarring of the choroid. The inventors have recognized that preservative-free or preservative-reduced compositions can be prepared by either reducing the concentration of the aforementioned preservatives by 1:10 to 1:10,000 (or any dilution between), or not including the aforementioned preservatives in the composition. In some embodiments, the preservative-free compositions of this disclosure can be made by the methods for preparing preservative-free opthalmological compositions described in WO2017182138 (also published as U.S. Pat. No. 11,229,596), incorporated by reference herein.

In some embodiments, the nitric-oxide releasing agents can be substantially encapsulated in an emulsion. As used herein, the term “emulsion” refers to a dispersed phase (e.g., the nitric oxide-releasing agent as described herein and any optionally additional pharmacologically active agent), a dispersion medium and an emulsifying agent. If desired, emulsion stabilizers can be included in the formulation as well. A number of pharmaceutically useful emulsions are known in the art, including oil-in-water (o/w) formulations, water-in-oil (w/o) formulations and multiple emulsions such as w/o/w or o/w/o formulations. The dispersion medium can be oil or water. The oil dispersion medium can include or exclude paraffin-based oils, plant oils, silicone oils, petroleum-based oils (e.g., mineral oil). Thus, the compositions comprising a nitric oxide-releasing agent of this disclosure can include or exclude an emulsifying agent. Emulsifying agents suitable for use in such formulations can include or exclude: TWEEN®60 (Polyethylene glycol sorbitan monostearate), Span 80® (sorbitan oleate), cetostearyl alcohol, myristyl alcohol, glyceryl monostearate, sodium lauryl sulfate, potassium laurate, sodium octane sulfonate, sodium decane sulfonate, sodium dodecane sulfonate, sodium lauryl sulfate, docusate sodium, decyltrimethylammonium bromide, dodecyltrimethylammonium bromide, tetradecyltrimethylammonium bromide, tetradecyltrimethyl-ammonium chloride, dodecylammonium chloride, polyoxyl 8 dodecyl ether, polyoxyl 12 dodecyl ether, nonoxynol 10 and nonoxynol 30.

In some embodiments, the nitric oxide-releasing agent can be substantially encapsulated within a microsphere. Microspheres, also referred to herein as “microparticles”, encapsulate a drug or drug-containing formulation. Microspheres can be formed from synthetic or naturally occurring biocompatible polymers. In some embodiments, the compositions comprising nitric oxide-releasing agents of this disclosure can include or exclude a microsphere. In some embodiments, microspheres suitable for encapsulating nitric oxide-releasing agents include those described in Lee et al., Journal of Pharmaceutical Investigation, Volume 37, Issue 2, 95-99 (2007); Abdel-Montaleb, European Journal of Pharmaceutics and Biopharmaceutics, Volume 79, Issue 1, September 2011, Pages 36-42; U.S. Pat. Nos. 8,969,415, 8,569,272, 8,697,044, and U.S. Patent Application Publication Nos. US20130189369, and US20050009910 each of which is herein incorporated by reference. Microspheres can also be selected from crosslinked PEG, PLGA, PLA, PCL, hyaluronic acid, polyacrylamide, cellulose, guar gum, locust bean gum, and combinations thereof.

Microspheres have a number average diameter of 1 to 100 μm, most preferably 1 to 25 μm. Microparticles may or may not be spherical in shape. Microspheres can be solid spheres, can be porous and include a sponge-like or honeycomb structure formed by pores or voids in a matrix material or shell, or can include multiple discrete voids in a matrix material or shell. The microparticle may further include a matrix material. The shell or matrix material may be a polymer, amino acid, saccharide, or other material known in the art of microencapsulation.

The nitric oxide-releasing agent-containing microparticles or nanoparticles may be suspended in an aqueous or non-aqueous liquid vehicle. The liquid vehicle may be a pharmaceutically acceptable aqueous solution, and optionally may further include a surfactant. The microparticles or nanoparticles of drug themselves may include an excipient material, such as a polymer, a polysaccharide, a surfactant, etc., which are known in the art to control the kinetics of drug release from particles.

In one embodiment, the compositions comprising a nitric oxide-releasing agent of this disclosure can include or exclude an agent effective to degrade collagen or GAG fibers in the sclera, which may enhance penetration/release of the drug into the ocular tissues. This agent may be, for example, an enzyme, such a hyaluronidase, a collagenase, or a combination thereof.

In some embodiments, the compositions comprising a nitric oxide-releasing agent of this disclosure can include or exclude viscosity modulating agents. In some embodiments, the viscosity of the compositions comprising a nitric oxide-releasing agent of this disclosure is controlled to maintain injectability (reducing back pressure in the injection apparatus) of the compositions during suprachoroidal injection. In some embodiments, the viscosity of the compositions comprising a nitric oxide-releasing agent of this disclosure are no greater than that of water. In some embodiments, the viscosity is no greater than 2.0, 1.9, 1.8, 1.7, 1.6, 1.5, 1.4, 1.3, 1.2, 1.1, or 1.0 mPas-sec. In some embodiments, the viscosity is no greater than 1 mPas-sec. Low viscosity solutions also increase ocular absorption of the active compound, decrease variability in dispensing the composition, and decrease physical separation of components of a suspension, liposome, or emulsion of the composition. The suprachoroidal injection is a potential space (not an anatomical space) in the eye so the viscosity of the composition is preferably close to that of water (1 mPas sec) so that the suprachoroidal space can be created with a fluid wave.

In some embodiments, the compositions comprising a nitric oxide-releasing agent of this disclosure can include or exclude a viscosity reducing agent. In some embodiments, the viscosity-reducing agent is selected from: 1-phenylalanine, thiamine phosphoric acid ester chloride dihydrate, benzenesulfonic acid, pyridoxine hydrochloride, aspartic acid, ethylenediaminetetraacetic acid, hydroxyethylethylenediaminetriacetic acid, and diethylenetriaminepentaacetic acid, carnosine, 2,4-diaminobutyric acid, ornithine, alendronic acid, glutathione, phosphocreatine; arginine, glutathione, and 2,4-diaminobutyric acid. In some embodiments, the viscosity-reducing agent is selected from a dipeptide selected from: H-His-Tyr-OH, H-His-Ala-OH, H-His-Gly-OH, H-His-Ser-OH, H-His-Phe-OH, H-His-Lys-OH, H-His-Arg-OH, H-HIS-Vsl-OH, H-Asp-Gly-OH, H-Hid-Asp-OH, H-Asp-Leu-OH, (as referred to in Proj, M. et al. Comput Struct Biotechnol J. 2022; 20: 5420-5429. published online 2022 Sep. 26. doi: 10.1016/j.csbj.2022.09.035; PMCID: PMC9529560PMID: 36212536). In some embodiments, the viscosity-reducing agent is selected from n-acetyl arginine, n-acetyl lysine, n-acetyl histidine, n-acetyl proline. N-acetyl arginine, n-acetyl lysine, n-acetyl histidine, and n-acetyl proline are modified versions of a naturally-occurring amino acids. N-acetyl arginine, n-acetyl lysine, n-acetyl histidine, and n-acetyl proline include both d and 1 forms of the amino acids, such as n-acetyl-1 arginine, n-acetyl-d arginine, n-acetyl-1 lysine, n-acetyl-d lysine, n-acetyl-1 histidine, n-acetyl-d histidine, n-acetyl-1 proline and n-acetyl-d proline (as described in U.S. Patent Application Publication No. 20170333559). Such agents are typically employed at a level from 0.1 to 5% by weight.

In some embodiments, the compositions comprising a nitric oxide-releasing agent of this disclosure can include or exclude suspending agents (e.g., polyvinyl pyrrolidone, glycerin monostearate, sorbitan esters, lanolin alcohols), dispersing agents (e.g., surfactants such as tyloxapol and polysorbate 80, ionic polymers such as sodium alginate), to ensure that the composition is dispersed in a uniform microparticulate suspension.

When the ophthalmic formulation is in the form of an aqueous suspension or solution, a non-aqueous suspension or solution, or a gel or ointment it is preferable to use a pH modifier to make the formulation have a pH between about 4 and 8, more preferably between about 6.8 to about 7.5. A preferred pH modifier is hydrochloric acid, sulfuric acid, boric acid, sodium hydroxide or any other ophthalmically-acceptable pH modifier.

In some embodiments, the compositions comprising a nitric oxide-releasing agent of this disclosure can include or exclude physiologic levels of serum electrolytes which modulates the osmolality of the formulation from about 200 to about 500 mOsm/Kg, preferably from about 250 to about 400 mOsm/Kg, and more preferably from about 280 to about 320 mOsm/Kg. Examples of osmolality excipients include, but are not limited to: dextrose, sodium chloride, potassium chloride, glycerin, various buffers and the like.

In some embodiments, the compositions comprising a nitric oxide-releasing agent of this disclosure can include or exclude from about 10 to about 1500 mg/L, and more preferred from about 700 to about 1250 mg/L of a nutrient sugar. In yet a further contemplation of the present invention the nutrient sugar of the formulation is a monosaccharide, oligosaccharide or polysaccharide. Examples of the nutrient sugars include, but are not limited to: dextrose, fructose, galactose, glucose, mannose, N-acetyl-galactosamine, N-acetyl-glucosamine, N-acetyl-neuraminic acid, and xylose. In yet still a further contemplation of the invention provides that the nutrient sugar of the present formulation is dextrose at a concentration from about 700 to about 1250 mg/L.

### Methods of Preparing the Compositions

In one embodiment, the composition comprising a nitric oxide-releasing agent is an ointment prepared by the following method:
(a) admixing a thickener and 80-90% of the total water amount in the final composition, and heating to a temperature of about 45-75° C. to provide a first solution,
(b) admixing the emulsifier, penetration enhancer, and the remaining portion of the water with heating to a temperature of about 35° C. to about 70° C. to provide a second solution,
(c) admixing the first solution and second solution at a lowered temperature, preferably below about 50° C. to provide an ointment base,
(d) admixing the ointment base with the nitrogen oxide releasing agent, the remaining portion of the penetration enhancer and optional ancillary ingredients to yield the ophthalmic ointment.

The ointment can be administered to the subject by topical application or injection to the eye of a subject.

In some embodiments, the ancillary ingredients can include or exclude a pH adjusting agent. The ideal pH of the pharmaceutical composition comprising nitric oxide-releasing agents of this disclosure are preferably about 6-8. One or more ophthalmically acceptable pH adjusting agents and/or buffering agents can be included. The acids can include or exclude: acetic, boric, citric, lactic, phosphoric and hydrochloric acids. The bases can include or exclude: sodium hydroxide, sodium bicarbonate, sodium carbonate, sodium phosphate, sodium borate, sodium citrate, sodium acetate, sodium lactate and tris-hydroxymethylaminomethane (TRIS). The buffers can include or exclude: citrate/dextrose, sodium bicarbonate/carbonate, phosphate-buffered saline, Ringer's solution, and ammonium chloride. Such acids, bases and buffers are included in an amount required to maintain pH of the composition in an ophthalmically acceptable range.

In some embodiments, the ancillary ingredients can include or exclude osmality adjusting salts (hypertonicity agents). One or more ophthalmically acceptable salts can be included in the composition in an amount required to bring osmolality of the composition into an ophthalmically acceptable range. Such salts can include or exclude: sodium, potassium, or ammonium cations; and chloride, citrate, ascorbate, borate, phosphate, bicarbonate, sulfate, thiosulfate or bisulfite anions. In some embodiments, the salts can include or exclude: sodium chloride, potassium chloride, sodium thiosulfate, sodium bisulfite, ammonium sulfate, and combinations thereof.

In some embodiments, the ancillary ingredients can include or exclude a buffer. The buffer can be an acetate buffer, citrate buffer, or phosphate buffer.

### Dosages, Amounts and Concentrations

The amount and/or concentration of the active nitric oxide-releasing agent(s) in any of the aforementioned dosage forms and compositions will depend on a variety of factors, including the type of dosage form, the corresponding mode of administration, the intended release profile, the nature and activity of any secondary active agents, the age and general condition of the subject being treated, the severity of the subject's condition, and other factors known to the prescribing physician.

Preferred dosage forms contain a unit dose of a nitric oxide-releasing agent, i.e., a single therapeutically effective dose. A “unit dose” requires an active agent concentration that provides a unit dose in a specified quantity of the formulation to be applied. In some embodiments, a “unit dosage” of nitroglycerin that is administered is in the range of about 0.05 mg to about 150 mg, typically about 0.1 mg to about 30 mg, preferably about 0.2 mg to about 20 mg, and most preferably about 0.2 mg to about 10 mg. The formulation may be administered on an as-needed basis, or on an ongoing basis, for example once, twice or three times daily. Preferably, the formulation is administered on an as-needed basis.

In a preferred embodiment, drug administration is on an as-needed basis, and does not involve chronic drug administration. That is, an immediate release dosage form may be used to administer the drug, such that substantially all of the drug (i.e., greater than 80% by weight, preferably greater than 90%) is released from the composition or dosage form within about 4 hours, preferably within about 2 hours, most preferably within about 1 hour, following administration. With a sustained release dosage form, a single dose can provide therapeutic efficacy over an extended time period in the range of about 1 to 20 days. In some embodiments, a single dose can provide therapeutic efficacy from 3 to 6 days, depending on the formulation. The release period may be varied by the selection and relative quantity of particular sustained release liposomes or microparticles encapsulating the nitric oxide-releasing agent, viscosity modulating agents, active ingredient compositions, pH, and ionic strength. If necessary, drug administration may be carried out within the context of an ongoing dosage regimen, i.e., on a weekly basis, twice weekly, daily, twice daily, etc.

When a plurality of nitric oxide-releasing agents, or a nitric oxide-releasing agent described herein is co-administered with a PDE5 inhibitor as described herein, are used to treat choroidal vascular diseases which can include or exclude central serous chorioretinopathy (CSC), diabetic or hypertensive choroidopathy, choroidal ischemia or choroidal neovascularization by the methods described herein, a therapeutically effective amount of a plurality of the nitric oxide-releasing agents and/or PDE5 inhibitor may be administered simultaneously, separately or sequentially and in any order. The agents may be administered separately or as a fixed combination. When not administered as a fixed combination, preferred methods include the sequential administration of one or more nitric oxide-releasing agents as described herein with a PDE5 inhibitor as described herein, either or both of which are provided in amounts or doses that are less that those used when the agent or agents are administered alone, i.e., when they are not administered in combination, either physically or in the course of treatment of a wound. Such lesser amounts of agents administered are typically from about one-twentieth to about one-tenth the amount or amounts of the agent when administered alone, and may be about one-eighth the amount, about one-sixth the amount, about one-fifth the amount, about one-fourth the amount, about one-third the amount, and about one-half the amount when administered alone. Preferably, the agents are administered sequentially within at least about one-half hour of each other. The agents may also be administered with about one hour of each other, with about one day to about one week of each other, or as otherwise deemed appropriate. Preferably, the nitric oxide-releasing agent or agents in a topical formulation is/are nitroglycerin, isosorbide dinitrate, isosorbide mononitrate, or combinations thereof. Oral application of both drugs (nitric oxide-releasing agent or agents and/or PDE5 inhibitor) simultaneously will lead to severe hypotension and is contraindicated. Therefore, the nitric oxide-releasing agent will be administered locally (e.g., by suprachoroidal injection or subtenon injection) and the PDE5 inhibitor administered systemically to avoid systemic hypotension.

The effective dose of the nitric oxide-releasing agent can be determined by routine experimentation or other methods known in the art or later developed. For example, in order to formulate a range of dosage values, cell culture assays and animal studies can be used. The dosage of such compounds preferably lies within the dose that is therapeutically effective for at least 50% of the population, and that exhibits little or no toxicity at this level.

In some embodiments, the therapeutically effective amount of the nitric oxide-releasing agent may be from about 0.001 to about 1 mg/kg body weight such as about 0.01 to about 0.4 mg/kg body weight. In some embodiments, the therapeutically effective amount of the nitric oxide-releasing agent may be from about 0.001 to about 0.1 mg/kg body weight such as about 0.01 to about 0.050 mg/kg body weight. Doses from about 1 to 100, 100-200, 200-300, 300-400, and 400-500 micrograms as well as 500-750 and 750-1000 micrograms are appropriate.

In some embodiments, the nitric oxide-releasing agent may be delivered using about 0.02 millimolar (mM) to about 100 millimolar (mM) final concentration at the treatment site and/or adjacent to the treatment site. Preferably, the nitric oxide releasing agent is applied at about 0.022 mM to about 88 mM final concentration. In certain other embodiments, the nitric oxide-releasing agent is applied at about 10 μM final concentration. In yet another embodiment, the nitric oxide-releasing agent composition is applied at about 1-15 μM final concentration. Nitric oxide-releasing agent dose amounts include, for example, about 0.01-1, 1-2, 2-3, 3-4, or 4-5 micrograms (g), from about 5 to about 10 μg, from about 10 to about 15 g, from about 15 to about 20 μg, from about 20 to about 30 g, from about 30 to about 40 g, from about 40 to about 50 g, from about 50 to about 75 g, from about 75 to about 100 μg, from about 100 μg to about 250 ag, and from 250 μg to about 500 ag. Dose amounts from 0.5 to about 1.0 milligrams or more or also provided, as noted above. Dose volumes will depend on the size of the site to be treated, and may range, for example, from about 25-100 μL to about 100-200 μL, from about 200-500 μL to about 500-1000 μL.

Still other dosage levels between about 1 nanogram (ng)/kg and about 1 mg/kg body weight per day of the agents described herein. In certain embodiments, the dosage of each of the subject compounds will generally be in the range of about 1 ng to about 1 microgram per kg body weight, about 1 ng to about 0.1 microgram per kg body weight, about 1 ng to about 10 ng per kg body weight, about 10 ng to about 0.1 microgram per kg body weight, about 0.1 microgram to about 1 microgram per kg body weight, about 20 ng to about 100 ng per kg body weight, about 0.001 mg to about 100 mg per kg body weight, about 0.01 mg to about 0.1 mg per kg body weight, or about 0.1 mg to about 1 mg per kg body weight. In certain embodiments, the dosage of each of the subject compounds will generally be in the range of about 0.001 mg to about 0.01 mg per kg body weight, about 0.01 mg to about 0.2 mg per kg body weight, about 0.1 mg to about 1 mg per kg body weight. If more than one nitric oxide-releasing agent is used, the dosage of each nitric oxide-releasing agent need not be in the same range as the other. For example, the dosage of one nitric oxide-releasing agent may be between about 0.01 mg to about 1 mg per kg body weight, and the dosage of another nitric oxide-releasing agent may be between about 0.1 mg to about 0.5 mg per kg body weight.

In some embodiments, the amount of the nitric oxide releasing agent administered, the period of administration, and the general administration regime may differ between subjects depending on such variables as the target site to which it is to be delivered, the severity of any symptoms of a subject to be treated, the type of disorder to be treated, size of unit dosage, the mode of administration chosen, and the age, sex and/or general health of a subject and other factors known to those of ordinary skill in the art.

Data obtained from cell culture assays and animal studies can be used in formulating a range of dosages for use in humans. The nitric oxide-releasing agent dosage may vary within this range depending upon the dosage form employed and the route of administration utilized. For any agent used in the method of the invention, the therapeutically effective dose can be estimated initially from cell culture assays. A dose may be formulated in cell cultures or animal models to achieve a cellular concentration range that includes the IC50 (i.e., the concentration of the test compound that achieves a half-maximal inhibition of symptoms) as determined in cell culture. Such information can be used to more accurately determine useful doses in humans. The exact formulation, route of administration and dosage can be chosen by the individual physician in view of the patient's condition. The dosage can be determined from the concentration of the amount administered, expected mass of the animal model tested (200-300 g per rat for adult Wistar rats), to determine the dose in units of mg/kg from concentration (micromolar) administered or amount (mg) administered.

Examples of effective doses that may be used for the treatment of vascular diseases including choroidal vascular diseases (which can include or exclude central serous chorioretinopathy, diabetic or hypertensive choroidopathy, choroidal ischemia or choroidal neovascularization) are described and claimed herein. In some embodiments, the therapeutically effective amount of the nitric oxide-releasing agent, which is effective to treat choroidal vascular diseases (which can include or exclude central serous chorioretinopathy, diabetic or hypertensive choroidopathy, choroidal ischemia or choroidal neovascularization) is a concentration of about 0.05 weight percent to about 4.0% weight percent of the overall composition. In some embodiments, the therapeutically effective amount of the nitric oxide-releasing agent is from 1.0 microgram/ml, or from about 0.001 to about 0.01 mg/ml, or from about 0.1 mg/mL to about 100 mg/mL, or more, or any range between any two of the recited dosages or any dose between any two recited numbers. The dose can be 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mg/ml or any range between any two of the recited dosages or any dose between any two recited numbers. In some embodiments, the therapeutically effective amount of the nitric oxide-releasing agent is present at a concentration ranging from about 0.05 to about 4.0% w/w (0.05-40 mg nitroglycerin/Ig of the composition). In some embodiments, the volume of distribution of nitroglycerin following intravenous administration is from 1 L/kg to 9 L/kg. In some embodiments, the volume of distribution of nitroglycerin following intravenous administration is about 3 L/kg. In some embodiments, tt plasma concentrations between 50-500 ng/mL, the binding of nitroglycerin to plasma proteins is approximately 60%. The In some embodiments, the half-life of nitroglycerin in plasma is about 3-5 minutes.

In some embodiments, the nitric oxide-releasing agent can be administered at a therapeutically effective dose between about 0.001 to about 100 mg/kg, between about 0.001 to about 0.01 mg/kg, between about 0.01 to about 0.1 mg/kg, between 0.1 to about 1 mg/kg, between about 1 to about 10 mg/kg, or between about 10 to about 100 mg/kg, or any range between any two recited dosages or any dose between any two recited dosages. In some aspects, the dose can be 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mg/ml or any range between any two of the recited dosages or any dose between any two recited numbers.

For each of the therapeutically effective concentrations, amounts or doses described above, the dose of a nitric oxide-releasing agent may be between 1/10 to 1/100, or between 1/100, to 1/1000 of any of the recited concentrations, amounts or doses, or any range between any two recited dosages or any dose between any two recited dosages.

It should be appreciated that administration may include a single daily dose, administration of a number of discrete divided doses, or continuous administration, as may be appropriate. By way of example, unit doses may be administered once or more than once per day, for example 1, 2, 3, 4, 5 or 6 times a day to achieve a desired total daily dose. By way of example, a unit dose of a nitric oxide-releasing agent may be administered in a single daily dose or a number of discrete doses, or continuously to achieve a daily dose of approximately 0.1 to 10 mg, 10 to 100 mg, 100 to 1000 mg, 1000 to 2000 mg, or 2000 mg to 5000 mg, 0.1 to approximately 2000 mg, approximately 0.1 to approximately 1000 mg, approximately 1 to approximately 500 mg, approximately 1 to approximately 200 mg, approximately 1 to approximately 100 mg, approximately 1 to approximately 50 mg, or approximately 1 to approximately 25 mg, or any range between any two recited dosages or any dose between any two recited dosages. In some embodiments, the single dose can be 0.1 mg. In some embodiments, the single dose can be 0.2 mg. In some embodiments, the single dose can be 0.3 mg. In some embodiments, the single dose can be 0.4 mg. In some embodiments, the single dose can be 0.5 mg. In some embodiments, the single dose can be 0.6 mg. In some embodiments, the single dose can be 0.7 mg. In some embodiments, the single dose can be 0.8 mg. In some embodiments, the single dose can be 0.9 mg. In some embodiments, the single dose can be 1.0 mg.

By way of further example, a unit dose of a nitric oxide-releasing agent may be administered once or more than once a day (for example 1, 2, 3, 4, 5 or 6, typically 1 to 4 times a day), such that the total daily dose is in the range (for a 70 kg adult) of approximately 1 to approximately 50 mg, for example approximately 1 to approximately 5 mg, or 5 mg to 10 mg, 10 to 20 mg, or 20 mg to 50 mg, or any range between any two recited dosages or any dose between any two recited dosages. For example, a nitric oxide-releasing agent may be administered to a subject at a dose range of approximately 0.01 to approximately 15 mg/kg/day, for example approximately 0.05 to approximately 2 mg/kg/day, for example approximately 0.07 to approximately 1 mg/kg/day, or any range between any two recited dosages or any dose between any two recited dosages.

In one embodiment, the dose of nitric oxide-releasing agent is approximately 0.001 micromolar to 0.1 micromolar, 0.1 micromolar and up to approximately 200 micromolar at the site of action, or higher, within the circulation to achieve those concentrations at the site of action. By way of example, the dose may be (but not limited to) a final circulating concentration of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 micromolar, or any range between any two recited concentrations, or any concentration between any two recited numbers.

The inventors have recognized that the suprachoroidal space is of limited volume—typically from 100 to 400 microliters (0.1 to 0.4 mL). The volume of the suprachoroidal injected nitric oxide releasing agents of this disclose can therefore be selected from 50 to 400 microliters for the dose. The volume of the suprachoroidal injected nitric oxide releasing agents of this disclose can therefore be selected from 50 to 100 microliters for the dose. In some embodiments, the dose of suprachoroidal injected nitric oxide releasing agents of this disclosure can have a volume selected 50, 55, 60, 65, 70, 75, 80, 85, 90, 95, 100, 105, 110, 115, 120, 15, 130, 135, 140, 145, 150, 155, 160, 167, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400 microliters, or any volume between the aforementioned volumes.

In one embodiment, the dose of a nitric oxide-releasing agent may be 10, 100 or 1000 fold lower than any of the recited doses set forth herein.

Conveniently, the nitric oxide-releasing agent is administered in a sufficient amount to effect vasodilation. The nitric oxide-releasing agent, for example, nitroglycerin, isosorbide mononitrate, or isosorbide dinitrate, is administered in a sufficient amount to achieve vasodilation for at least about 0.2 to 1 hour, at least about 1-2 hours, at least about 2-4 hours, at least about 4-6 hours, at least about 6-8 hours, at least about 8-10 hours, at least about 12 hours, or at least about 24 hours post-administration.

The doses of the composition comprising the nitric oxide-releasing agent may be administered in single or divided applications. The doses may be administered once, or application may be repeated. Typically, application will be repeated weekly, biweekly, or every 3 weeks, every month, or every 2, 3, 4, 5, 6, 7, 8, 9, 10, 11, 12, 13, 14, 15, 16, 17, 18, 19, 20, 21, 22, 23, or every 24 months or more as needed to prevent, slow, or treat ocular neuropathy, or any ocular condition described herein. The dose may be repeated, and/or increased or decreased in the event that neuronal loss increases or decreases. Doses may also be applied every 12 hours to 7 days apart, or more. For example, doses may be applied 12 hours, or 1, 2, 3, 4, 5, 6, or 7 days apart, or at any time interval falling between any two of these times, or between 12 hours and 7 days. The nitric oxide-releasing agent may be administered for up to four, six, eight, ten, twelve, fourteen, sixteen, eighteen, twenty, twenty-two, twenty-four or twenty-six weeks. For some indications, such as certain ocular uses, more frequent dosing, up to hourly may be employed. In some embodiments, the doses will be administered as a series of weekly injections for 3-4 weeks when the subject is a human.

### Kits

Provided herein are kits comprising a 0.05-4% percent by weight a nitric oxide-releasing agent (including nitroglycerin), a container, directions for use, a measuring device, and optionally an applicator. In some embodiments, the applicator is selected from an eye applicator or a syringe with needle. In some embodiments, the needle can be a microneedle. In some embodiments, the measuring device can be within the applicator, e.g., volumetric indicia within the applicator.

In some embodiments, a “kit” or “article of manufacture” refers to a combination of items as described above. In some embodiments, the kit comprises a container comprising a composition comprising a nitric oxide-releasing agent. The kit may further comprise a label or package insert, on or associated with the container. The term “package insert” is used to refer to instructions customarily included in commercial packages of therapeutic products, that contain information about the indications, usage, dosage, administration, contraindications and/or warnings concerning the use of such therapeutic products. Suitable containers include, e.g., bottles, vials, syringes, blister pack, etc. The container may be formed from a variety of materials such as glass or plastic. The label or package insert indicates that the composition is used for treating the choroidal vascular disease of choice, which can include or exclude central serous chorioretinopathy The label or package insert may also indicate that the composition can be used to treat other disorders. The kit may further comprise directions for the administration of the composition comprising a nitric oxide-releasing agent.

In some embodiments, the kit may further comprise a third container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

In some aspects, the first and second (and optionally, third) compositions of the kit can be administered in combination, can be administered simultaneously, can be administered separately, can be administered sequentially, or can be administered in a sustained manner.

### EXAMPLES

The following non-limiting examples further illustrate pharmaceutical compositions and dosage forms of the present invention.

### Example 1

| | |
|---|---|
| Water | 84.0% |
| Nitroglycerin | 2.0 |
| Propylene Glycol | 4.0 |
| Glycerin | 3.0 |
| Mineral Oil | 2.0 |
| Potassium Phosphate | 1.0 |
| Lidocaine | 2.0 |
| Isopropyl Palmitate | 1.0 |
| Polyoxyl Stearate | 1.0 |

### Example 2

| | |
|---|---|
| Water | 82.0% |
| Nitroglycerin | 1.0 |
| Propylene Glycol | 8.0 |
| Lidocaine | 4.0 |
| Polyethlylene Glycol | 1.0 |
| Methylcellulose | 1.0 |
| Lidocaine | 2.0 |
| Isopropyl Palmitate | 1.0 |

### Example 3

| | |
|---|---|
| Water | 83.0% |
| Nitroglycerin | 0.5 |
| Propylene Glycol | 6.0 |
| Lidocaine | 2.0 |
| Polyethlylene Glycol | 4.0 |
| Methylcellulose | 1.0 |
| Laurocapram | 2.0 |
| Fragrance | 0.5 |
| Isopropyl Palmitate | 1.0 |

### Example 4

| | |
|---|---|
| White Petrolatum | 88.0% |
| Nitroglycerin | 3.0 |
| Lidocaine | 3.0 |
| Polyethlylene Glycol | 1.0 |
| Mineral oil | 4.0 |
| Sodium Chloride | 1.0 |

### Example 5

| | |
|---|---|
| White Petrolatum | 86.0% |
| Nitroglycerin | 2.0 |
| Mineral oil | 5.0 |
| Lidocaine | 2.0 |
| Polyethylene Glycol | 1.0 |
| Hydroxyethylcellulose | 3.0 |
| Benzalkonium Chloride | 1.0 |

### Example 6

| | |
|---|---|
| White Petroleum | 90% |
| Heavy Liquid Paraffin | 5 |
| Nitroglycerin | 2 |
| Lidocaine | 1.0 |
| Hydroxypropylmethylcellulose | 0.5 |
| Carbomer | 0.5 |
| Benzalkonium Chloride | 1.0 |

### Example 7

| | |
|---|---|
| White Petroleum | 80% |
| Heavy Liquid Paraffin | 10 |
| Nitroglycerin | 2 |
| Lidocaine | 2 |
| Hydroxypropylmethylcellulose | 2 |
| Carbomer | 2 |
| Benzalkonium Chloride | 2 |

### Example 8

| | |
|---|---|
| White Petroleum | 75% |
| Heavy Liquid Paraffin | 17 |
| Nitroglycerin | 3 |
| Lidocaine | 2 |
| Hydroxypropylmethylcellulose | 1 |
| Carbomer | 1 |
| Benzalkonium Chloride | 1 |

As a representative method, the aqueous components of each of the Examples 1-8 are first combined, then optionally filtered with a 0.22 micron Supor filters into depyrogenated, sterilized (by autoclaving) vials. Only sterile water is used in Examples 1-3. Next, the emulsifiers (if present) are added and the solutions mixed until clear. The hydrophobic components are separately premixed and then added to the aqueous phase and the solutions mixed until clear. Components shown above are protected from light and air to minimize oxidation and degradation.

In some embodiments, the Examples 1-8 can be pH adjusted using a buffer selected from PBS, Ringer's solution, sodium bicarbonate, sodium hydroxide, and citric acid (and their corresponding salts).

In some embodiments, the Examples 1-8 can be degassed (freeze-pump-thaw or subject to vacuum) prior to use.

### Example 9. Method of Treating Subjects Having Central Serous Chorioretinopathy with Compositions of the Invention

The nitric-oxide releasing agent isosorbide dinitrate was given orally at 5 mg twice per day dosage for sublingual administration for 7 days for 7 human patients (CL, WZ, XC, LL, BZ, XZ, and LS) having central serous chorioretinopathy (CSCR). Average age of the patients is 41.6 years. 5 men and 2 women were enrolled in this study. As shown in Table 1, the central macular OCT (Optical coherence tomography) thickness shows a generalized decrease of CSC after 7-10 days after treatment, thereby demonstrating the clinical effectiveness of treating CSC with a nitric oxide-releasing agent of this disclosure.

TABLE 1
OCT data of 7 subjects treated with nitric oxide-releasing agent

| | Baseline macular | Post-treatment | % |
|---|---|---|---|
| Patient/ | thickness by | macular thickness | increase(+)/ |
| Eye | OCT (um) | by OCT (um) | decrease(−) |
| CL OD | 315 | 255 | −19.0% (all |
| | | | fluid resolved) |
| CL OS | 488 | 295 | −39.5% |
| WZ OS | 231 | 173 | −25.1% |
| XC OD | 467 | 321 | −31.3% |
| LL OD | 256 | 234 | −8.6% |
| BZ OD | 453 | 315 | −30.5% |
| XZ OD | 304 | 288 | −5.3% |
| LS OS | 259 | 178 | −31.3% |

### Example 10. Method of Treating Subjects Having Central Serous Chorioretinopathy with Ophthalmic Compositions of the Invention

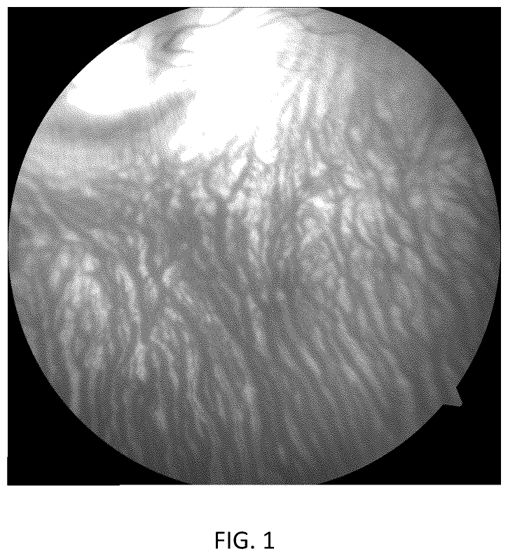

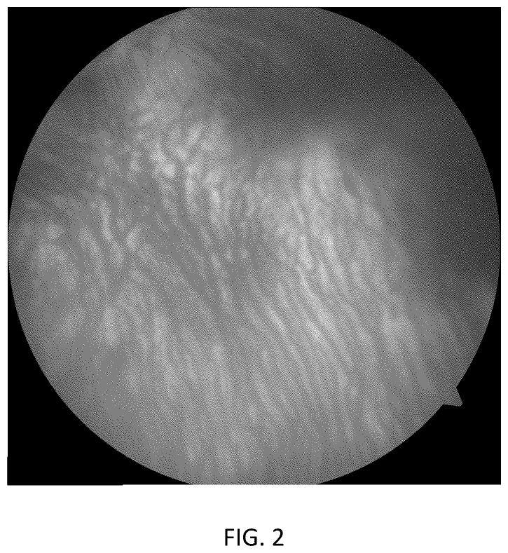

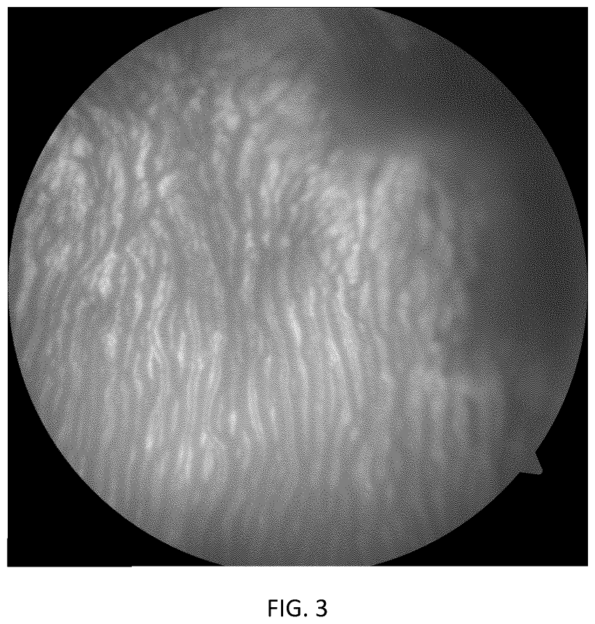

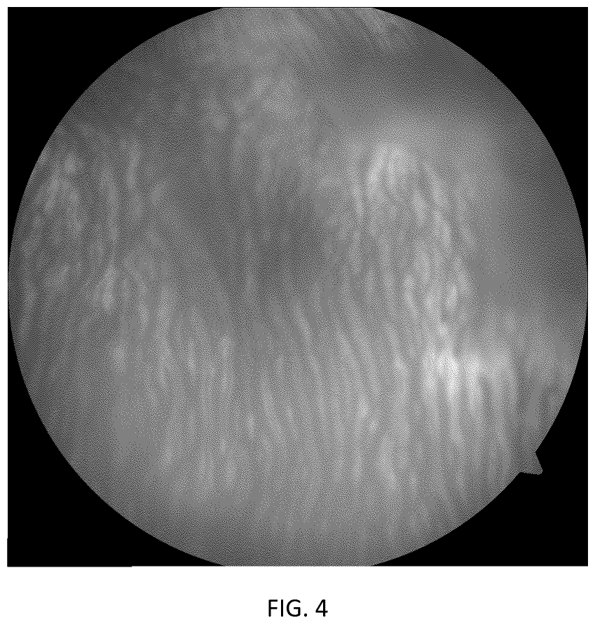

Ophthalmologic compositions comprising a nitric oxide-releasing agent for administration to the eye were prepared by the methods described herein. Briefly, 0.5 mg/0.1 mL nitroglycerin (in buffered saline as a fluid form) was administered into the suprachoroidal space of a New Zealand White rabbit using the Clearside microinjector. As the rabbits are albinos, the choroidal vessels were clearly visible. As shown in FIGS. 1-4, the amount of hyperemia (small pink micro-vessels in between the larger choroidal vessels) of the choroid after the suprachoroidal injection is clearly remarkable at 10, 20 and 30 min time intervals post-injection relative to baseline (pre-injection). This microvascular choroidal dilation is a model for treating central serous chorioretinopathy where leakages are caused by focal blockages of the micro-capillaries.

It is expected that the macular thickness can be measured by OCT (optical coherence tomography), and is expected to show a decrease in OCT by over 10% relative to baseline macular thickness, indicating that local administration of the ophthalmologic compositions comprising a nitric oxide-releasing agent described herein can treat vascular diseases including choroidal vascular diseases (which can include or exclude central serous chorioretinopathy (CSC), diabetic and hypertensive choroidopathy, choroidal ischemia and choroidal neovascularization).

### Example 11. Preparation of Representative Liposomal Composition

As a representative example for preparing a suspension composition comprising a nitric oxide releasing agent of this disclosure wherein the nitric oxide releasing agent is nitroglycerin, a composition can be prepared as follows: 40 mg of nitroglycerin with 0.55% (weight/volume [w/v]) sodium chloride for tonicity, 0.5% (w/v) carboxymethylcellulose sodium, and 0.02% (w/v) polysorbate 80. It also contains potassium chloride, calcium chloride (dihydrate), magnesium chloride (hexahydrate), sodium acetate (trihydrate), sodium citrate (dihydrate), and water for injection. Hydrochloric acid may be used to adjust pH to a target value of 6.5.

The inventions described and claimed herein have many attributes and embodiments including, but not limited to, those set forth or described or referenced in this Detailed Disclosure. It is not intended to be all-inclusive and the inventions described and claimed herein are not limited to or by the features or embodiments identified in this Detailed Disclosure, which is included for purposes of illustration only and not restriction. A person having ordinary skill in the art will readily recognize that many of the components and parameters may be varied or modified to a certain extent or substituted for known equivalents without departing from the scope of the invention. It should be appreciated that such modifications and equivalents are herein incorporated as if individually set forth. The invention also includes all of the steps, features, compositions and compounds referred to or indicated in this specification, individually or collectively, and any and all combinations of any two or more of said steps or features.

All patents, publications, scientific articles, web sites, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Applicants reserve the right to physically incorporate into this specification any and all materials and information from any such patents, publications, scientific articles, web sites, electronically available information, and other referenced materials or documents. Reference to any applications, patents and publications in this specification is not, and should not be taken as, an acknowledgment or any form of suggestion that they constitute valid prior art or form part of the common general knowledge in any country in the world.

The specific methods and compositions described herein are representative of preferred embodiments and are exemplary and not intended as limitations on the scope of the invention. Other objects, embodiments, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, in embodiments or examples of this, any of the terms “comprising”, “consisting essentially of”, and “consisting of” may be replaced with either of the other two terms in the specification. Also, the terms “comprising”, “including”, containing”, etc. are to be read expansively and without limitation. The methods and processes illustratively described herein suitably may be practiced in differing orders of steps, and that they are not necessarily restricted to the orders of steps indicated herein or in the claims. It is also that as used herein and in the appended claims, the singular forms “a,” “an,” and “the” include plural reference unless the context clearly dictates otherwise. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants. Furthermore, titles, headings, or the like are provided to enhance the reader's comprehension of this document, and should not be read as limiting the scope of this. Any examples of embodiments, embodiments or components of the invention referred to herein are to be considered non-limiting.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although this has been specifically disclosed by preferred embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or embodiments of the invention are described in terms of Markush groups, those skilled in the art will recognize that the invention is also thereby described in terms of any individual member or subgroup of members of the Markush group.

What is claimed:

1. A method for treating an ocular vascular disease in a subject in need thereof, said method comprising administering to the subject a dosage of a therapeutically effective amount of a composition comprising a nitric oxide-releasing agent.

2. The method of claim 1, wherein the ocular vascular disease is of the choroid.

3. The method of claim 1, wherein the administering to the subject comprises contacting the sclera or the conjunctiva of the subject with said nitric oxide-releasing agent.

4. The method of claim 3, wherein the contacting the sclera or the conjunctiva is performed by subtenon injection to the eye of said subject.

5. The method of claim 3, wherein the contacting the sclera or the conjunctiva is performed by topical application to the eye of said subject.

6. The method of claim 1, wherein the administering is performed by suprachoroidal injection to the eye of said subject.

7. The method of claim 1, wherein the ocular vascular disease is selected from central serous chorioretinopathy (CSC) or diabetic choroidopathy, hypertensive choroidopathy, choroidal ischemia, dry ARMD, wet ARMD and choroidal neovascularization.

8. The method of claim 7, wherein the ocular vascular disease is central serous chorioretinopathy.

9. The method of claim 8, whereby choroidal vessel dilation occurs after administration of the nitric-oxide releasing agent is administered.

10. The method of claim 1, wherein the nitric oxide-releasing agent is selected from isosorbide dinitrate, isosorbide mononitrate, nitroglycerin (1,2,3-Tris(nitrooxy)propane), or a combination thereof.

11. The method of claim 1, further comprising administering to the subject a therapeutically effective amount of a phosphodiesterase 5 inhibitor.

12. The method of claim 11, wherein the phosphodiesterase 5 inhibitor is selected from sildenafil (Viagra), tadalafil (Cialis), avanafil, vardenafil (Levitra), and pharmaceutically acceptable salts thereof.

13. The method of claim 1, wherein the dosage of the composition comprises 0.05-4% by weight, based upon the total weight of said composition, of said nitric oxide-releasing agent.

14. The method of claim 1, wherein said composition further comprises an effective amount of an anesthetic.

15. The method of claim 14, wherein said anesthetic is present in an amount from about 0.1% to about 10% by weight, based upon the total weight of said composition.

16. The method of claim 14, wherein said anesthetic is a topical anesthetic.

17. The method of claim 16, wherein the topical anesthetic is selected from proparacaine, dibucaine, pramoxine, prilocaine, dibucaine, benzocaine, tetracaine lidocaine, tetracaine, rodocaine (N-(2-chloro-6-methylphenyl)octahydro-trans-1H-pyrindine-1-propanamide), bupivacaine, benoxinate, and combinations thereof.

18. The method of claim 1, wherein said composition is adapted for topical application over the conjunctiva or sclera of the eye.

19. The method of claim 1, wherein said composition is adapted for suprachoroidal injection to the eye.

20. The method of claim 1, wherein said composition further comprises one or more pharmaceutically acceptable carriers or excipients in an admixture with said nitric oxide-releasing agent.

21. The method of claim 19, wherein said excipients comprise white petrolatum, canola oil, mineral oil, lanolin, parrafin wax, distilled water, acetone sodium bisulfite, zinc oxide, cocoa butter, or mixtures thereof.

22. The method of claim 19, wherein the composition further comprises a liposome and wherein the nitric oxide-releasing agent is substantially located within the liposome.

23. The method of claim 1, wherein said composition is administered to the eye at least one time daily.

24. The method of claim 23, wherein the composition is administered 2-8 times daily.

25. The method of claim 1, wherein said composition is administered to the bulbar conjunctiva of the eye.

26. A method of treating central serous chorioretinopathy in a subject in need thereof, said method comprising administering by suprachoroidal injection to the subject a dosage of a therapeutically effective amount of a composition comprising a nitric oxide-releasing agent selected from isosorbide dinitrate, isosorbide mononitrate, nitroglycerin (1,2,3-Tris(nitrooxy)propane), or a combination thereof.
